(19) United States
(12) Patent Application Publication
(10) Pub. No.: US 2010/0160436 A1
(43) Pub. Date: Jun. 24, 2010

(54) STABLE GABAPENTIN COMPOSITIONS

(75) Inventors: Vincenzo CANNATA, Sasso Marconi (IT); Francesco Corcella, Rozzano (IT); Andrea Nicoli, Vicenza (IT)

Correspondence Address: OBLON, SPIVAK, MCCLELLAND MAIER & NEUSTADT, L.L.P., 1940 DUKE STREET, ALEXANDRIA, VA, 22314, US

(73) Assignee: ZaCh System S.p.A., Bresso (IT)

(21) Appl. No.: 12/714,920

(22) Filed: Mar. 1, 2010

Related U.S. Application Data
(63) Continuation of application No. 12/190,090, filed on Aug. 12, 2008.
Application No. 12/190,090 is a continuation of application No. 11/774,892, filed on Jul. 9, 2007, now abandoned.
Application No. 11/774,892 is a continuation of application No. 11/336,846, filed on Jan. 23, 2006, now abandoned.
Application No. 11/336,846 is a continuation of application No. 10/024,339, filed on Dec. 21, 2001, now abandoned.

(51) Int. Cl. A61K 31/195; C07C 225/20; A61P 25/28; A61P 25/16; A61P 25/08; A61P 25/22; A61P 25/00

(52) U.S. Cl. 514/561; 562/507

(57) ABSTRACT

Stable compositions containing gabapentin compositions, methods of preparing such compositions, and methods of using such compositions.

### BACKGROUND OF THE INVENTION

1. Field of the Invention

The present invention relates to stable gabapentin compositions. The present invention also relates to methods of preparing these compositions and to methods of using these compositions.

2. Description of the Background

Gabapentin (1-aminomethyl)cyclohexaneacetic acid) is a well-known therapeutic for treating and improving a variety of neurological/cerebral conditions and also improve cerebral functions. Examples of such conditions include epilepsy, faintness attacks, hypokinesia, cranial traumas, as described in, for example, U.S. Pat. No. 4,024,175.

It is also known in the art that gabapentin is difficult to formulate due to, inter alia, formation of the intramolecular lactam derivative (hereinafter referred to as “gabapentin lactam”). Various methods have been described to reduce the tendency of gabapentin to form gabapentin lactam in the bulk material and in final, unit dosage forms. For example, U.S. Pat. No. 6,054,482 describes a method of preparing gabapentin which is free of the gabapentin lactam. These gabapentin compositions contain less than 20 ppm of an anion of a mineral acid

However, these methods are not entirely satisfactory for producing gabapentin of high purity. Accordingly, there remains a need in the art for stable gabapentin compositions.

### SUMMARY OF THE INVENTION

It is an object of the present invention to provide stable gabapentin-containing compositions.

It is another object of the present invention to provide compositions containing more than 20 ppm of an anion of a mineral acid, e.g., chloride.

It is another object of the present invention to provide methods of preparing these compositions.

It is another object of the present invention to provide methods of treating a variety of conditions using such compositions.

Accordingly, the objects of the invention, and others, may be accomplished with a composition comprising gabapentin and at most 5 ppm, based on the amount of the gabapentin, of an addition salt of gabapentin and an acid.

The objects of the invention may also be accomplished with a pharmaceutical composition in dry unit dosage form, comprising:

(a) gabapentin;

(b) at most 5 ppm, based on the amount of the gabapentin, of an addition salt of gabapentin and an acid; and

(c) at least one nonacidic pharmaceutically acceptable excipient.

The objects of the invention may also be accomplished with A pharmaceutical composition in dry unit dosage form, comprising:

(a) gabapentin;

(b) at least one salt of a nonacidic cation and an anion of a mineral acid, and

(d) at least one nonacidic excipient

wherein the composition contains at least 20 ppm of the anion of a mineral acid, based on the amount of the gabapentin.

The objects of the present invention may also be accomplished with methods of treating various disorders using the gabapentin compositions described above.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1: stability of gabapentin compositions as described in Example 1 herein;

FIG. 2: stability of gabapentin compositions as described in Example 2 herein; and

FIG. 3: stability of gabapentin compositions as described in Example 3 herein.

FIG. 4: stability of gabapentin compositions as described in Example 4 herein.

### DETAILED DESCRIPTION OF THE INVENTION

### Preparation of Gabapentin

Methods of synthesizing gabapentin are well-known in the art. Gabapentin can be prepared using any of these synthetic procedures. Preferably, the gabapentin is prepared using one of the synthetic procedures described in U.S. Pat. No. 4,024,175. Most preferably, the gabapentin is synthesized via the Hofmann rearrangement described in U.S. Pat. No. 4,024,175. Such a process produces a solution of the hydrochloride salt of gabapentin. This material may then be extracted or crystallized to produce a gabapentin solution containing 5 and 10 molar % of sodium chloride. This solution may then be dissolved in water and applied to a column filled with a strong cation exchange resin. Examples of such resins include IRA 120, DIAION SK 18, and IMAC HP 1110.

The gabapentin and sodium ions are first fixed to the resin using water as the eluant and the chloride and any residual organic solvents are removed using water. Next, the resin is eluted with an aqueous ammonia solution to release the gabapentin. The ammonia solution preferably has a concentration equal to or less than 4%. The released gabapentin can be released isolated by techniques well-known in the art, e.g., evaporation and subsequent crystallization. In a preferred embodiment of the present invention, the gabapentin is produced as polymorph form 1.

### Content of Acid Addition Salt of Gabapentin

In one embodiment, the composition of the present invention contains gabapentin and at most 5 ppm, based on the amount of the gabapentin, of an acid addition salt of gabapentin and an acid (hereinafter referred to as “the acid addition salt”).

The most relevant acid addition salt is gabapentin hydrochloride, i.e., the salt of gabapentin and hydrochloric acid. However, the acid may be another mineral acid such as hydrobromic acid, hydroiodic acid, phosphoric acid, nitric acid, sulfuric acid, sulfonic acid, or methanesulfonic acid.

The amount of the acid addition salt may be lower than 5 ppm, such as 4, 3, 2, 1, 0.5, 0.25, 0.1, 0.05 ppm or less.

It is particularly preferred that the composition contains an undetectable amount of the addition salt of hydrochloric acid in a silver nitrate titration assay. This assay may be performed by potentiometrically titrating with 0.01 N aqueous silver nitrate a solution obtained by dissolving 7.5 grams of the composition in 100 mL of methanol/water (80/20 by volume) followed by acidification with nitric acid. This assay is well-known to those skilled in the art.

### Salt of an Nonacidic Cation and an Anion of a Mineral Acid

In another embodiment, the composition of the present invention gabapentin and at least one salt of a nonacidic cation and an anion of a mineral acid, wherein the composition comprises more than 20 ppm of the anion of a mineral acid, based on the amount of the gabapentin. As used herein, the term “nonacidic cation” refers to a cation that is not a Bronsted or a Lewis acid. Thus, the amount of the anion of the mineral acid is higher than 20 ppm, such as 25, 30, 40, 50, 75, 100, 250, 500, 1000, 2000, 2500, 3000 ppm, or more.

Such a composition may be prepared, for example, by adding one or more salts of of a nonacidic cation and an anion of a mineral acid to the gabapentin produced with less than 5 ppm of the acid addition salt as described above.

The composition may also be prepared by adding the appropriate amount of the nonacidic cation hydroxide salt (e.g., NaOH) to a sample of gabapentin containing more than 20 ppm of chlorides in order to transform the existing chlorides into a salt with the nonacidic cation (e.g., NaCl).

In one embodiment, the composition additionally comprises at most 5 ppm of one or more addition salts of gabapentin and an acid. The amount of the acid addition salt may be lower than 5 ppm, such as 4, 3, 2, 1, 0.5, 0.25, 0.1, 0.05 ppm or less.

In one embodiment, the nonacidic cation is selected from the group consisting of alkali metals and alkaline earth metals. Suitable examples of such metals include lithium, sodium, potassium, magnesium, and calcium.

In one embodiment, the nonacidic cation is selected from the group consisting of quaternary ammonium groups. Suitable quaternary ammonium groups include tetraalkyl ammonium groups.

In another embodiment, the anion anion of a mineral acid is selected from the group consisting of fluoride, chloride, bromide, iodide, sulfate, and phosphate.

A preferred anion is chloride. A particularly preferred salt is sodium chloride.

### Unit Dosage Forms

The compositions containing gabapentin in bulk form as described above may be formulated into pharmaceutically acceptable unit dosage forms. Such unit dosage forms are well-known in the art. Aceptable unit dosage forms include tablets, caplets, and capsules.

Formulation processes which use a minimum amount of water are preferred. Such processes include dry tableting and anhydrous tableting procedures. These procedures are well-known to those skilled in the art and are described in, for example, in Kirk-Othmer Encyclopedia of Chemical Technology, Volume 18, Fourth Edition, pp. 480-510, incorporated herein by reference.

### Additives for Unit Dosage Forms

The additives for the unit dosage forms, i.e., pharmaceutically acceptable excipients, of the present invention are those which minimize the transformation of gabapentin to the corresponding lactam. Preferably, the excipients are nonacidic. As used herein the term “nonacidic excipient” refers to excipients that are not protic, Bronsted, or Lewis acids. The amount of ecipients may vary over a wide range. For example, the excipients may comprise 0.5 to 95% by weight of the unit dosage form.

### Water Content

The water content in the compositions of the present invention is preferably as low as possible. It is particularly preferred that the gabepentin be anhydrous. The water content is preferably at most 1% by weight in the bulk material and unit dosage forms. This range for the amount of water includes all specific values and subranges therebetween, such as at most 0.5, 0.2, 0.15, 0.12, 0.10, 0.05, 0.01, 0.01% by weight, or even less.

### Lactam Content

Due to its reported toxicity, the amount of gabapentin lactam in the compositions of the present invention is preferably as low as possible. The lactam content is preferably at most 0.5% by weight in the bulk material and unit dosage forms. This range for the amount of lactam includes all specific values and subranges therebetween, such as at most 0.4, 0.3, 0.2, 0.15, 0.10, 0.08, 0.05, 0.04, 0.02, 0.01, or even less.

### Methods of Use

The compositions of the present invention may be used in all of treatment methods using gabapentin which are known in the art. Accordingly, the compositions of the present invention may be used in such treatment methods. In each case, an effective amount of the composition is administered to a subject. Preferably, the subject is a human.

Thus, the present invention method of treating a cerebral disease. Examples of the cerebral disease include epilepsy, faintness attacks, hypokinesia, dizziness, and cranial trauma.

The present invention also includes a method of improving cerebral function. In this embodiment of the invention, the subject may be a geriatric patient.

The present invention also includes a method of treating a neurodegenerative disorder. Examples of the neurodegenerative disorder include stroke, Alzheimer's disease, Huntington's disease, Amyotrophic Lateral Sclerosis, and Parkinson's disease.

The present invention also includes a method of treating depression or anxiety.

The present invention also includes a method of treating or preventing panic attacks.

In addition, the present invention also includes a method of treating headaches.

For a detailed description of such methods, see U.S. Pat. Nos. 4,024,175, 5,025,035, 5,084,479, 5,792,796, each of which is incorporated herein by reference.

### EXAMPLES

Having generally described this invention, a further understanding can be obtained by reference to certain specific examples which are provided herein for purposes of illustration only and are not intended to be limiting unless otherwise specified.

### Example 1

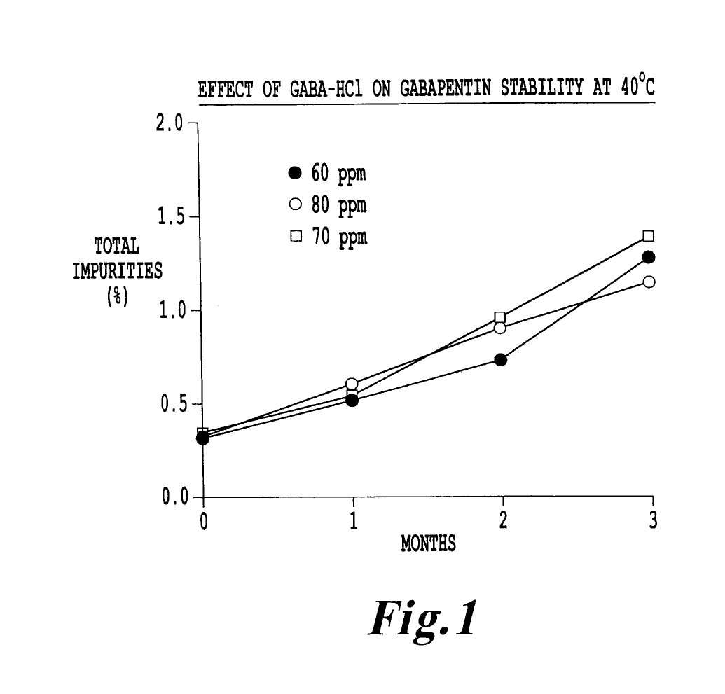

The stbility of a gabapentin compositions containing 60, 70, or 80 ppm of gabapentin hydrochloride (GABA-HCl) was measured at 40° C. by HPLC over a period of 3 months. The total amount of impurities was measured. The results are shown in FIG. 1.

### Example 2

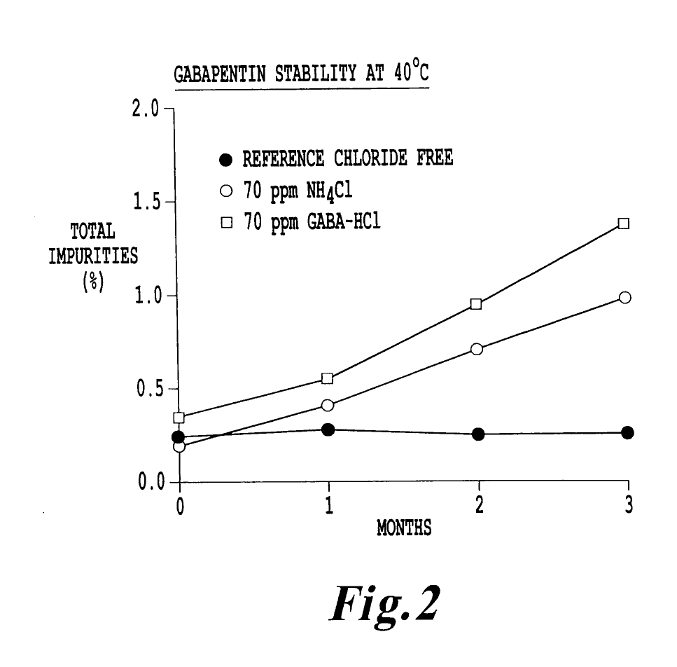

The stbility of a gabapentin compositions which was chloride free, i.e., no GABA-HCl, contained 70 ppm of NH₄Cl or 70 ppm of GABA-HCl was measured at 40° C. by HPLC over a period of 3 months. The total amount of impurities was measured. The results are shown in FIG. 2.

### Example 3

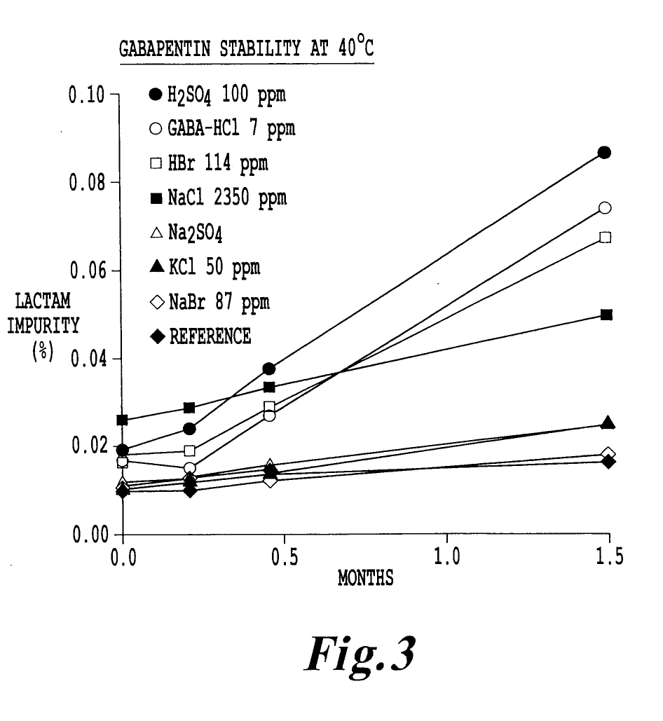

The stability of gabapnetin compositions, with respect to gabapentin lactam formation, at 40° C. containing (1) no additives (free of salts; denoted reference), (2) 87 ppm of NaBr, (3) 50 ppm of KCl, (4) 50 ppm of Na₂SO₄, (5) 2350 ppm of NaCl, (6) 114 ppm of HBr, (7) 7 ppm of GABA-HCl, or (8) 100 ppm of H₂SO₄. The amount of gabapentin lactam produced over 1.5 months was deteremined. The results are shown in FIG. 3. The results of this experiment demonstrated that gabapentin compositions containing a salt of a nonacidic cation were quite stable.

### Example 4

In order to prepare a composition of gabapentin containing 60 ppm of chloride (as NaCl), gabapentin (dry, 330 g), demineralized water (165 g), and methanol (218 g) were charged into a 2000 mL reactor. Isopropanol (915 g) was added dropwise under stirring to the mixture which was stirred at 50° C. The resulting mixture was stirred at 50° C. for a further 15 minutes and then cooled at −5° C. The resulting solid was then filtered and washed on the filter with 330 g of a NaCl solution in isopropanol/water (308 ppm). The product was then dried in an oven at 50° C. for 17 hours. As a result, gabapentin (307.5 g) was obtained with a chloride content of 60 ppm as measured by potentiometric titration with AgNO₃. Gabapentin containing different amounts of chloride (as NaCl), e.g., 70 or 80 ppm, can be prepared in a similar fashion.

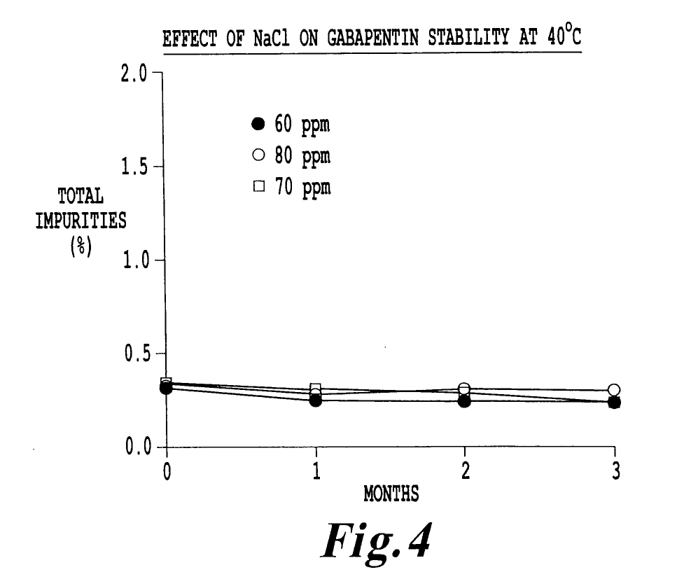

In order measure the stability of such compostions, samples prepared as described above containing (1) 60 ppm of chloride as NaCl, (2) 80 ppm of chloride as NaCl, and (3) 60 ppm of chloride as NaCl (duplicate) were stored at 40 C and anlyzed by HPLC to determine the amount of degredation product formed (including gabapentin lactam). The results of this experiment are presented in FIG. 4.

This experiment deomonstrates that gabapentin compositions may contain more, in fact, much more, of an anion of a mineral acid, e.g., chloride, and remain stable, provided that the counter-ion to the chloride is a nonacidic cation, e.g., sodium ion. This is in direct contrast to the teaching of U.S. Pat. No. 6,054,482, which teaches that the content of an anion of a mineral acid in a gabapentin composition must be less than 20 ppm. This experiment demonstrates that no meaningful degredation occurs and the amount of gabapentin lactam remains close to zero.

Obviously, numerous modifications and variations of the present invention are possible in light of the above teachings. It is therefore to be understood that within the scope of the appended claims, the invention may be practiced otherwise than as specifically described herein.

Unless stated otherwise above, all publications cited herein are incorporated herein by reference.

1-18. (canceled)

19. A composition, comprising gabapentin and at least one salt of a nonacidic cation and an anion of a mineral acid, wherein said composition comprises more than 20 ppm of said anion of a mineral acid, based on the amount of the gabapentin, and wherein:
said composition comprises at most 0.5% by weight of gabapentin lactam;
said nonacidic cation is selected from the group consisting of alkali metals, alkaline earth metals, and mixtures thereof; and
said anion of a mineral acid is selected from the group consisting of fluoride, chloride, bromide, iodide, sulfate, phosphate, and mixtures thereof.

20. The composition of claim 19, which comprises more than 25 ppm of said anion of a mineral acid, based on the amount of the gabapentin.

21. The composition of claim 19, which comprises more than 30 ppm of said anion of a mineral acid, based on the amount of the gabapentin.

22. The composition of claim 19, which comprises more than 50 ppm of said anion of a mineral acid, based on the amount of the gabapentin.

23. The composition of claim 19, which comprises more than 75 ppm of said anion of a mineral acid, based on the amount of the gabapentin.

24. The composition of claim 19, which comprises more than 100 ppm of said anion of a mineral acid, based on the amount of the gabapentin.

25. The composition of claim 19, which comprises more than 250 ppm of said anion of a mineral acid, based on the amount of the gabapentin.

26. The composition of claim 19, which comprises more than 500 ppm of said anion of a mineral acid, based on the amount of the gabapentin.

27. The composition of claim 19, which comprises more than 1000 ppm of said anion of a mineral acid, based on the amount of the gabapentin.

28. The composition of claim 19, which comprises more than 2000 ppm of said anion of a mineral acid, based on the amount of the gabapentin.

29. The composition of claim 19, which further comprises at most 5 ppm of one or more addition salts of gabapentin and an acid.

30. (canceled)

31. The composition of claim 19, wherein said nonacidic cation is selected from the group consisting of lithium, sodium, potassium, magnesium, calcium, and mixtures thereof.

32-33. (canceled)

34. The composition of claim 19, wherein said anion of a mineral acid is selected from the group consisting of fluoride, chloride, bromide, iodide, sulfate, phosphate, and mixtures thereof.

35. The composition of claim 19, wherein said anion of a mineral acid is chloride.

36. The composition of claim 19, wherein the salt of a nonacidic cation and an anion of a mineral acid comprises sodium chloride.

37. The composition of claim 19, which is in dry unit dosage form.

38-55. (canceled)

56. A method of treating a cerebral disease, comprising administering an effective amount of the composition of claim 19 to a subject in need thereof.

57. The method of claim 56, wherein said cerebral disease is epilepsy, faintness attacks, hypokinesia, dizziness, or cranial trauma.

58. A method of improving cerebral function, comprising administering an effective amount of the composition of claim 19 to a subject in need thereof.

59. The method of claim 58, wherein said subject is a geriatric patient.

60. A method of treating a neurodegenerative disorder, perinatal, comprising administering an effective amount of the composition of claim 19 to a subject in need thereof.

61. The method of claim 60, wherein said neurodegenerative disorder is stroke, Alzheimer's disease, Huntington's disease, Amyotrophic Lateral Sclerosis, or Parkinson's disease.

62. A method of treating depression, comprising administering an effective amount of the composition of claim 19 to a subject in need thereof.

63. A method of treating anxiety, comprising administering an effective amount of the composition of claim 19 to a subject in need thereof.

64. A method of treating or preventing panic attacks, comprising administering an effective amount of the composition of claim 19 to a subject in need thereof.

65-73. (canceled)
